(19) United States
(12) Patent Application Publication
(10) Pub. No.: US 2004/0045053 A1
(43) Pub. Date: Mar. 4, 2004

(54) Pollen specific promoter

(76) Inventors: Andrew James Greenland, Bracknell (GB); Hilary Joan Rogers, Cardiff (GB); Patrick Joseph Hussey, Egham (GB)

Correspondence Address: SYNGENTA BIOTECHNOLOGY, INC., PATENT DEPARTMENT, 3054 CORNWALLIS ROAD, P.O. BOX 12257, RESEARCH TRIANGLE PARK, NC, 27709-2257, US

(21) Appl. No.: 10/310,542

(22) Filed: Dec. 5, 2002

Related U.S. Application Data
(63) Continuation of application No. 09/601,828, filed on Oct. 13, 2000, now abandoned.
Application No. 09/601,828 is a 371 of international application No. PCT/GB99/00232, filed on Jan. 22, 1999.

(30) Foreign Application Priority Data
Feb. 20, 1998 (GB) 9803661.9
Feb. 20, 1998 (GB) 9803660.1

(51) Int. Cl. A01H 1/00; C12N 15/82; C07H 21/04

(52) U.S. Cl. 800/284; 800/279; 536/23.2; 435/468; 435/320.1

(57) ABSTRACT

A recombinant nucleic acid which comprises a promoter sequence of the ZmC5 gene in maize, or a variant or fragment thereof, which acts as a promoter in pollen.

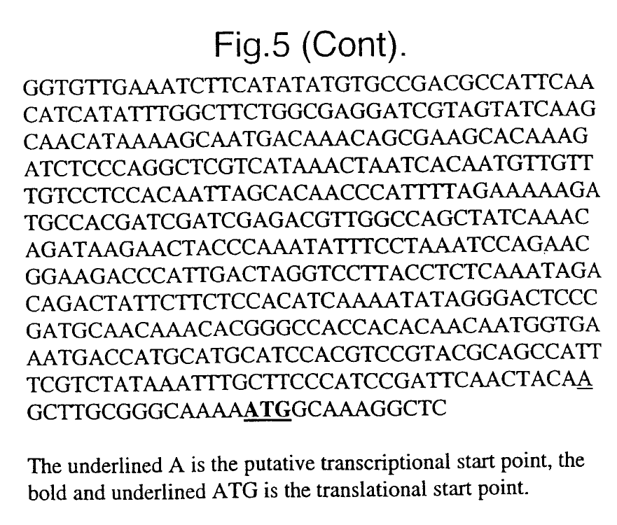

The DNA sequence of the promoter is depicted in FIG. 5. Expression cassettes and expression systems as well as transformation methods, transformed plants including the promoter sequence of the invention are also claimed. The nucleic acid may be used inter alia in the production of male sterile plants and/or hybrids as well as in the transformation of pollen.

The present invention relates to a promoter sequence which is specific for pollen, to constructs and transgenic plant cells and plants comprising the promoter as well as to methods of transforming pollen and controlling fertility in plants using this promoter.

In order to introduce desirable genetic traits from two plants into a single plant, such as a variety or hybrid, cross-breeding represents the traditional approach. In order to reliably obtain consistent hybrids, it is necessary to ensure that the self-pollination of the parent plants does not take place.

This can be achieved by ensuring that one of the parent lines is male sterile. Various techniques for producing male sterility are known and have been proposed in the art. One method involves removal of the anthers or tassels of the female parent plant, either manually or mechanically. This plant may then only be fertilised by pollen from the male parent and therefore its progeny will be hybrid. However, such a process is labour intensive and not altogether reliable as it is possible that some female plants may miss the detasseling process or in some cases, the plants develop secondary tassels after detasseling has been completed. In addition in this system the male parent is able to self pollinate so it is necessary to physically separate male and female parents to allow ease of harvest of hybrid seed. This block planting works well for corn as the pollen is light and produced in large quantity. However, this approach is not applicable to species with heavier pollen and in species in which the male and female floral organs are contained within one flower e.g. wheat, rice.

Chemical methods of preventing pollen production are also known. U.S. Pat. No. 4,801,326 describes chemicals which may be applied to the plant or soil to prevent pollen production. However, again such techniques are labour intensive and are not wholly reliable. It is essential that sufficient chemical is applied at the appropriate time intervals to ensure pollination does not occur, with the concomitant burden on the environment. In addition, these chemicals are very expensive.

Genetic engineering has provided an alternative way by which male sterility can be produced, maintained and/or subsequently restored in plants.

Systems have been described in which inducible promoters which allow fertility to be switched on or off depending upon the application or availability of an external compound. For example, WO 90/08830 describes the induction of male sterility in a plant by a cascade of gene sequences which express a protein which disrupts pollen biosynthesis. WO 93/18171 describes the use of a GST promoter to inducibly express chalcone synthase (chs) and restore fertility to a male sterile plant made sterile by “knocking out” the endogenous chs genes.

A different approach was described in WO931/125695 (PGS). This relies upon the use of a tapetum specific promoter to express a pollen lethality gene, barnase, in the tapetal cells which are critical to pollen development thus disrupting pollen production. A restorer gene, barstar, can be used to restore fertility (Mariani et al. Nature Vol 357: 384-387).

There would be advantages to expressing genes which have an impact in pollen production in pollen directly in a controllable manner. Suitably, fertility should be restorable when desired.

A component of a system to genetically engineer male sterility is the availability of a promoter which specifically drives expression either in pollen or in tissue on which pollen production or development depends.

Many of the characterised genes which are specifically or highly expressed in pollen and germinating pollen encode proteins that are likely to play a role in cell wall metabolism, for example, those having homology to genes encoding enzymes involved in pectin degradation; polygalacturonases (S M Brown et al.., Plant Cell (1990) 2: 263-274, S J Tebbutt et al.., Plant Mol. Biol. (1994) 25:283-299), pectate lyase (H J Rogers et al.., Plant Mol Biol 20:493-502 (1992), R A Wing et al, Plant Mol Biol 14:17-28 (1989)) and pectin methylesterase (PME) J H Mu et al.., Plant Mol Biol 25:539-544 (1994)).

Other genes highly expresed in pollen include those that encode cytoskeletal proteins (I. Lopez et al.., Proc. Natl, Acad Sci USA 93: 7415-7420 (1996), H J Rogers et al.., Plant J. 4: 875-882 (1993) and C J Staiger et al.., Plant J. 4: 631-641 (1993)), putative ascorbate oxidases, a Kunitz protein inhibitor and many others whose function cannot be inferred by homology to known genes. The temporal expression of such genes has been studied and many are found to be expressed late in microsporogenesis reaching a maximum in mature microsporocytes. In some cases continued expression in the pollen tube has also been demonstrated (A K Kononowicz et al.., Plant Cell 4; 513-524(1992)). These genes are referred to as “late genes”. The majority of expression at this stage is from the vegetative cell rather than from the generative cell and it is likely that the majority of these “late” genes are transcribed from the vegetative nucleus: although this has only been demonstrated for one “late” gene (D. Twell, Plant J2: 887-892 (1992). A distinct class of genes expressed in anthers is found to have a different expression programme, being first detectable soon after the tetrad stage and declining in expression well before pollen maturity. It is likely that the major role of these “early” genes may be during microspore differentiation and development rather than pollen tube growth. In addition, U.S. Pat. No. 5,086,169 (Mascarenhas) describes the isolation of the first pollen-specific promoter from corn.

The applicants have isolated a further promoter which is specifically expressed only in pollen tissue. The promoter is derived from a “late” pollen expressed gene isolated from maize, ZmC5.

Thus, according to a first aspect of the present invention there is provided a recombinant nucleic acid sequence which comprises a promoter sequence of the ZmC5 gene in maize, or a variant or fragment thereof, which acts as a promoter in pollen.

As used herein, the term “fragment” includes one or more regions of the basic sequence which retain promoter activity. Where the fragments comprise one or more regions, they may be joined together directly or they may be spaced apart by additional bases.

The expression “variant” with reference to the present invention means any substitution of, variation of, modification of, replacement of, deletion of or the addition of one or more nucleotides from or to the nucleic acid sequence providing the resultant sequence exhibits pollen promoter expression. The term also includes sequence that can substantially hybridise to the nucleic acid sequence.

As used herein, the expression “ZmC5 gene” refers to the gene of maize which encodes a 563 amino acid sequence as described herein. A cDNA sequence encoding this sequence is defined in EMBL Y13285

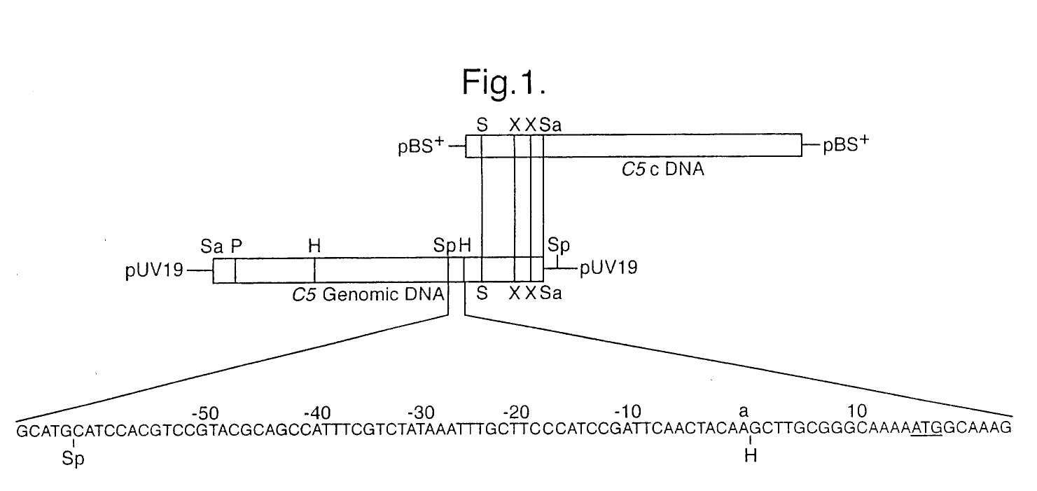

The promoter sequence of the present invention is comprised within the clone deposited National Collection of Industrial and Marine Bacteria as NCIMB 40915 on Jan. 26, 1998. This is a Sal I fragment derived as described hereinafter. The promoter region lies within a region which consists of approximately 2 kb of sequence upstream of the transcription start site of the ZmC5 gene of maize as shown in FIG. 1 hereinafter.

According to a preferred embodiment of the present invention, there is provided a recombinant nucleic acid sequence which comprises a promoter sequence comprising at least part of the DNA sequence as shown in FIG. 5 or at least part of a sequence encoding a promoter which has substantially similar activity to the promoter encoded by FIG. 5 or a variant or fragment thereof.

The term “substantially similar activity” includes DNA sequences which are complementary to and hybridise to the DNA of the present invention and which code for a promoter which acts in pollen. Preferably, such hybridisation occurs at, or between, low and high stringency conditions. In general terms, low stringency conditions can be defined as 3× SCC at ambient temperature of between about 60° C. to about 65° C., and high stringency conditions as 0.1× SSC at about 65° C. SSC is the name of a buffer of 0.15M NaCl, 0.01 5M trisodium citrate. 3× SSC is three times as strong as SSC and so on.

The pollen specific promoter of the present invention may be used to engineer male sterility by driving genes capable of interfering with pollen production or viability, or to express genes of interest specifically in pollen grains.

According to a second aspect of the present invention, the promoter sequence may form part of an expression cassette in combination with genes whose expression in pollen, and particularly in late pollen production, may be desirable. These include genes which have an impact on pollen or pollen production. Such genes may be those involved in the control of male-fertility, genes which encode insecticidal toxins (which would then be targeted to insect species which feed on pollen), or genes which would enhance or modify the nutritional value of the pollen. In addition, the promoter sequence could be used to drive expression of a selectable marker for use in pollen transformation. Examples of suitable selectable marker genes include antibiotic resistance genes such as kanamycin resistance gene, hygromycin resistance gene and the PAT resistance gene so as to enable stable transformants to be identified depending on the species e.g. corn, rice, wheat.

The term “expression cassette”—which is synonymous with terms such as “DNA construct”, “hybrid” and “conjugate”—includes an effect gene directly or indirectly attached to the regulator promoter, such as to form a cassette. An example of an indirect attachment is the provision of a suitable spacer group such as an intron sequence intermediate the promoter and the target gene. The DNA sequences may furthermore be on different vectors and are therefore not necessarily located on the same vector. The same is true for the term “fused” in relation to the present invention which includes direct or indirect attachment Such constructs also include plasmids and phage which are suitable for transforming a cell of interest.

According to a preferred embodiment, expression cassettes of the present invention comprise a promoter sequence as described above which is arranged to control expression of a gene which is deleterious to pollen development, such as genes encoding barnase, adenine nucleotide translocator, mutant tubulins, T-urf (as claimed in WO 97/04116) or trehalose phosphate phosphatase (TPP).

For instance, WO93/25695 describes the use of the gene barnase which encodes a cytotoxic protein , which is under the control of a tapetum specific promoter. Expression of barnase in the tapetal cells disrupts these cells and leads to disruption of pollen production.

Ribozymes are RNA molecules capable of catalysing endonucleolytic cleavage reactions. They can catalyse reactions in trans and can be targeted to different sequences. They are therefore potential alternatives to antisense as a means of modulating gene expression. (Hasselhof and Gerlach (1988) Nature Vol 334: 585-591) or Wegener et al. (1994) Mol Gen Genet 245: (465-470) have demonstrated the generation of a trans-dominant mutation by expression of a ribozyme gene in plants. If required, the pollen specific promoter of the present invention may be used to control expression of the ribozymes such that they are specifically expressed in pollen.

Baulcombe (1997) describes a method of gene silencing in transgenic plants via the use of replicable viral RNA vectors (Amplicons™) which may also be useful as a means of knocking out expression of endogenous genes. This method has the advantage that it produces a dominant mutation i.e. is scorable in the heterozygous state and knocks out all copies of a targeted gene and may also knock out isoforms. This is a clear advantage in wheat which is hexaploid. Fertility could then be restored by using an inducible promoter to drive the expression of a functional copy of the knocked out gene. By including the pollen specific promoter as the elements of the Amplicon™ m vector, expression of the gene would then take place specifically in the pollen.

The use of cytotoxic or disrupter genes as means of disrupting pollen production requires the expression of restorer genes to regain fertility. Suitably the construct further comprises a cassette comprising a nucleotide sequence which is able to overcome the effect of said deleterious gene, such as a restorer gene such as barstar in the case of barnase or TPS in the case of TPP, or a sequence which encodes a construct which is sense or antisense to a deleterious gene.

An alternative means of controlling expression of deleterious genes is to use operator sequences. Operator sequences such as lac, tet, 434 etc. may be inserted into promoter regions as described in WO 90/08830. Repressor molecules can then bind to these operator sequences and prevent transcription of the downstream gene, for example a gene deleterious to pollen development (Wilde et al. (1992) EMBO J. 11, 1251). Furthermore, it is possible to engineer operator sequences with enhanced binding capacity such as the Lac IΔ His mutant as described in (Lehming et al. (1987) EMBO . 6, 3145-3 153). This has a change of amino acid from tyrosine to histidine at position 17 thus giving tight control of expression. Used in combination with inducible expression of the repressor this then allows expression of an inactivating gene to be turned off.

In this way, the expression cassettes may be incorporated into expression systems which may be used in the control of fertility of a plant as described above.

The term “expression system” means that the system defined above can be expressed in an appropriate organism, tissue, cell or medium. The system may comprise one or more expression cassettes and may also comprise additional components that ensure to increase expression of the target gene by use of the regulator promoter.

According to a third aspect of the present invention, there is provided an expression system comprising

(a) a first promoter sequence which is expressed specifically in pollen;

(b) a first gene which, when expressed, disrupts pollen biogenesis, under the control of said pollen specific promoter;

(c) a second promoter sequence responsive to the presence or absence of an exogenous chemical inducer: and

(d) a second gene encoding an element which can inhibit either expression of said first gene. or can inhibit the protein coded for by said first gene, operably linked to and under the control of said second promoter sequence.

Elements (a) and (b) and (c) and (d) above may be provided by one or two individual vectors, but preferably are contained in the same vector to ensure co-segregation. These can be used to transform or co-transform plant cells so as to allow the appropriate interaction between the elements to take place.

The second promoter sequence and the second gene provide for chemical “switching” on and off of the first gene. Where the second promoter sequence is responsive to the presence of the exogenous chemical inducer, application of the chemical inducer to pollen or to a plant will have the effect of switching on the second gene which thereby counteracts the effect of the first gene. The absence of the chemical inducer will have a similar effect where the second promoter sequence is active only in the absence of the chemical inducer.

Elements (c) and (d) are suitably in the form of an expression cassette comprising a nucleotide sequence which is able to overcome the effect of said deleterious gene, such as a restorer gene such as barstar in the case of barnase or TPS in the case of TPP, or a sequence which encodes a construct which is sense or antisense to a deleterious gene, or a gene encoding a repressor molecule in the case of operator sequences being used operatively interconnected with an inducible promoter.

The expression system of the present invention may further comprise a selectable marker, such as herbicide resistance genes or antibiotic resistance genes so as to allow stable transformants to be identified depending on the species eg corn, rice, wheat. The presence of a herbicide resistance gene also allows selection of male sterile progeny in a segregating population.

Transformation of a plant with such an expression system will result in the production of male sterile plants and methods of producing such a plant form a fourth aspect of the present invention.

Expression systems in accordance with this embodiment of the present invention, wherein the gene is deleterious to viable pollen production, are useful in the production of hybrids but are especially useful when the male sterile line can be made homozygous. When “late” promoters, such as the ZmC5 promoter described above, are used, because the gene products are expressed late in pollen development, primary transformants and heterozygotes produce pollen which segregates 1:1 for sterility i.e. 50% of the pollen is fertile and so self pollination leading to non-hybrid seed may occur.

In order to obtain a homozygous sterile plant, the inducible promoter must be switched on to drive expression of the restorer gene using the appropriate chemical such as ethanol in the case of the AlcA/R switch or safener for the GST switch. This then inactivates the deleterious gene, so allowing self pollination to occur in accordance with the model below.

| MS = Dominant male sterility | | | |
|---|---|---|---|
| Rcs = inducible restorer | | | |
| Heterozygous genotype | | MSRcs--- | |
| Gametes | | MSRcs | --- |
| Self pollination | after chemical induction gives | | |
| | | MSRcs | --- |
| | MSRcs | MSMSRcsRcs | ---MSRcs |
| | --- | MSRcs-- | --- --- |

All pollen from the homozygous progeny (MSMSRcsRcs) will be sterile, 50% of the pollen from the heterozygous progeny (MSRcs---) will be sterile. All of the pollen from the null (--- ---) progeny will be fertile. Staining of the pollen from these lines with a vital stain such as DAPI will allow identification of the plant producing 100% sterile pollen. Alternatively, the induction and self pollination can be repeated on this segregating population and the progeny analysed for segregation of sterility. Clearly all progeny deriving from self pollination of the homozygous line will themselves be homozygous and male sterile i.e. they will all produce 100% sterile pollen, whereas progeny arising from self pollination of a heterozygous line will continue to segregate for sterility. Alternatively, if the gene giving rise to male sterility is linked to a gene conferring herbicide resistance, then progeny may be sprayed with herbicide at an early seedling stage, herbicide tolerance will segregate with the sterility gene. In this way, male sterile parents can be identified and selected for hybrid production.

Once identified, this homozygous sterile line may be used in the production of F1 hybrids by cross pollination by an unmodified inbred male parent line. See below.

| | |
|---|---|
| Female parent | Male parent |

| | |
|---|---|
| MSMSRcsRcs | *** *** |

| | | |
|---|---|---|
| Gametes | MSRcs | *** |
| MSRcs | MSRcs | |

| | | | |
|---|---|---|---|
| | *** | MSRcs*** | MSRCs*** |
| F1 | | | |
| | *** | MSRcs*** | MSRCS*** |

Thus, all the F1 seed is hybrid and heterozygous for sterility, meaning that 50% of the pollen from each plant is fertile. In a crop species such as corn this is ample viable pollen to ensure complete pollination across a field due to the sheer volume of pollen produced by each tassel. In wheat too this should be sufficient pollen to ensure no yield loss as self pollination occurs while the flower is still closed, thus reducing loss by wind etc. In species in which the vegetative part of the plant is harvested then reduced pollen viability is not a factor to be taken into account.

There is too an additional benefit to the hybrid seed producer in that should the farmer retain F2 seed for subsequent planting he will suffer a loss in yield as result of loss of heterosis. See below.

| F1 | | MSRcs*** | |
|---|---|---|---|
| Gametes | | MSRcs (not viable) *** | |
| | MSRcs | none | *** MSRcs |
| F2 | | | |
| *** | none | *** *** | |

These methods may allow for reversal of the sterility, for example in hybrid production, by activation using an inducible promoter.

According to a fifth aspect of the present invention, there is provided a method of controlling the fertility of a plant which comprises transforming said plant with an expression system as described above, and when fertility is to be restored, activating the inducible promoter.

Suitable inducible promoters include those which are controlled by the application of an external chemical stimulus, such a herbicide safener. Examples of inducible promoters include, for example, a two component system such as the alcA/alcR gene switch promoter system described in our published International Publication No. WO 93/21334, the ecdysone switch system as described in our International Publication No. WO 96/37609 or the GST promoter as described in published International Patent Application Nos. WO 90/08826 and WO 93/031294, the teachings of which are incorporated herein by reference. Such promoter systems are herein referred to as “switch promoters”. The switch chemicals used in conjunction with the switch promoters are agriculturally acceptable chemicals making this system particularly useful in the method of the present invention.

If the pollen specific promoter of the present invention is used to obtain male sterility, full restoration of fertility may not be achievable by this method as pollen is haploid. This means that only 50% of pollen produced following activation of the restorer gene is fertile. This may be countered by the fact that expression using the promoter of the present invention is highly tissue specific.

This property makes the use of the promoter of the present invention particularly useful in some very particular applications. For example, in some cases transformation of pollen is required. In this instance the use of the pollen specific promoter be may highly desirable. An example of such an application is known as MAGE LITER (male germ line transformation) and is described by Stoger et al (Plant Cell Reports 14 (1995) 273-278). In this method, pollen is transformed by microprojectile bombardment. A pollen specific promoter is used to drive, for example a selectable marker gene. The fact that the promoter is pollen specific confers several advantages. First of all, the marker is expressed only in the pollen, not the rest of the plant and so the remaining plant tissue does not contain unwanted marker. Furthermore, having a selectable marker only in the pollen allows the possibility of retransforming by the usual methods and not having to have a different selectable marker, i.e. it allows easier gene stacking. The reasons why pollen transformation is important are that pollen can be made to undergo sporophytic development i.e. will give rise to haploid and doubled haploid plants. This means that homozygosity is achieved in one step. Alternatively, the transformed pollen can be used to pollinate wildtype plants thus giving seed carrying the introduced transgene, again a faster process than the traditional transformation route.

The expression systems of the present invention can be introduced into a plant or plant cell via any of the available methods including infection by Agrobacterium tumefaciens containing recombinant Ti plasmids, electroporation, microinjection of plant cells and protoplasts, microprojectile bombardment, bacterial bombardment, particularly the “fibre” or “whisker” method, and pollen tube transformation, depending upon the particular plant species being transformed. The transformed cells may then in suitable cases be regenerated into whole plants in which the new nuclear material is stably incorporated into the genome. Both transformed monocot and dicot plants may be obtained in this way. Reference may be made to the literature for full details of the known methods. Such methods form a further aspect of the present invention.

The method of the present invention would be useful in the production of a wide range of hybrid plants, such as wheat, rice, corn, cotton, sunflower, sugar beet, and lettuce, oil seed rape and tomato.

Plant cells which contain a plant gene expression system as described above, together with plants comprising these cells form further aspects of the invention.

According to a nineth aspect of the present invention there is provided a replicable viral DNA vector (Amplicon™) which comprises a recombinant nucleic acid as defined above.

According to a tenth aspect of the present invention, there is provided a method of transforming pollen cells with an expression system as described above.

The invention will now be particularly described only by way of example with reference to the accompanying drawings in which:

FIG. 1 is a diagram showing the alignment of the ZmC5 cDNA with a 2.4 kb fragment from the 5′ region of its corresponding gene. The transcriptional start point is indicated (*), and the putative translational start is underlined. Sa=Sal I, S=Sma I, Sp=Sph I, H=Hind III, X=Xho I, P=Pst I;

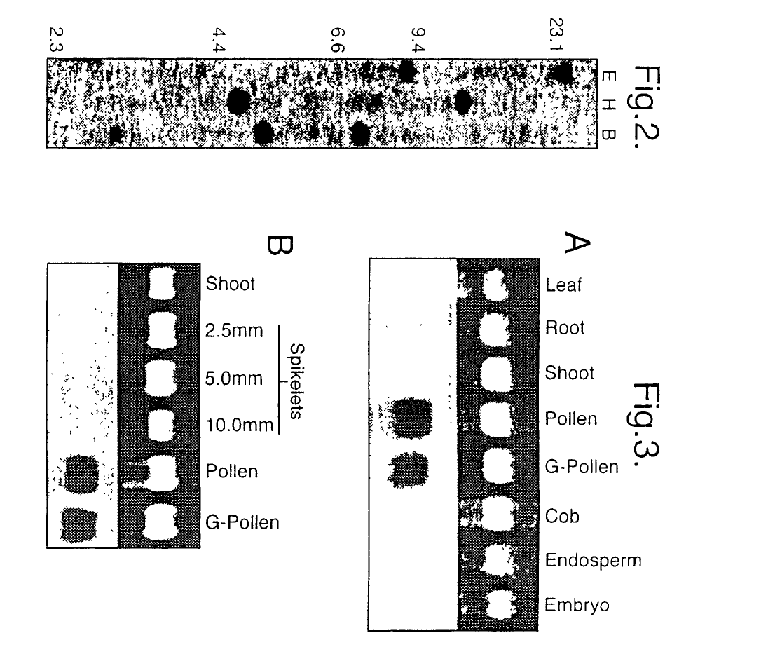

FIG. 2 shows a Southern blot of maize (inbred line A188) genomic DNA. Each lane contains 15 μg of genomic DNA digested in lane 1 with Eco RI, in lane 2 with Hind III, and in lane 3 with Bam HI. The Southern blot was hybridised with radiolabelled ZmC5 cDNA probe.

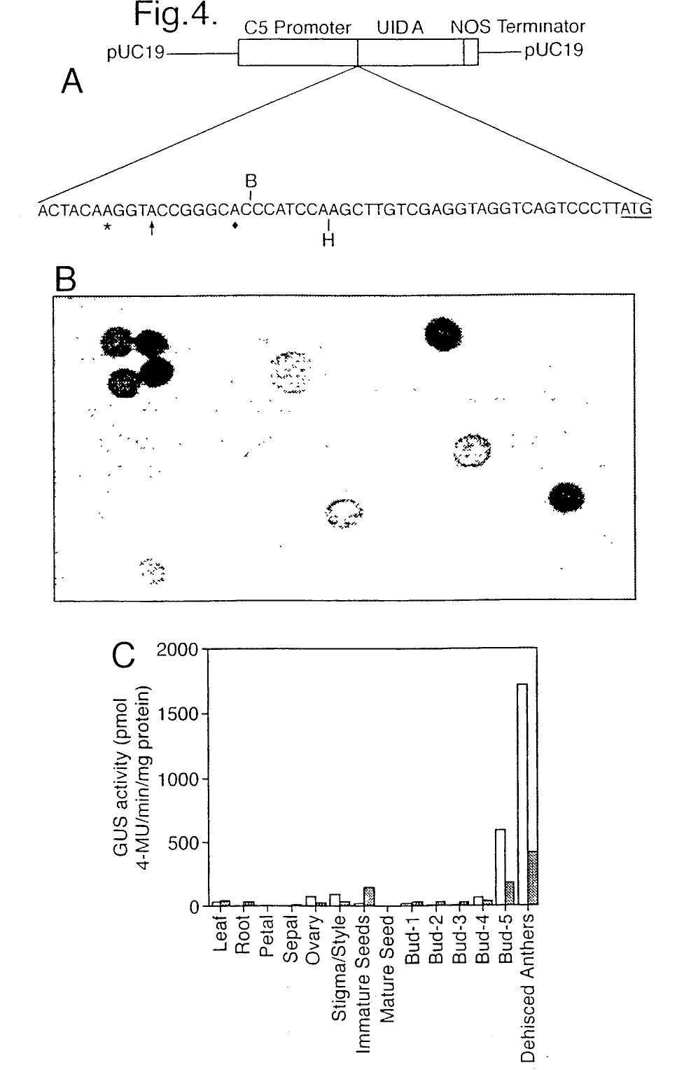

FIG. 3 shows ethidium bromide stained gels showing the 18S RNAs of various maize tissue total RNAs and the northern blots of the same gels probed with the ZmC5 cDNA probe.

(A) the gel was loaded with 10 μg of total RNA from various maize tissues.

(B) the gel was loaded with 10 μg of total RNA isolated from shoot, a developmental staged series of spikelets, pollen and germinating pollen.

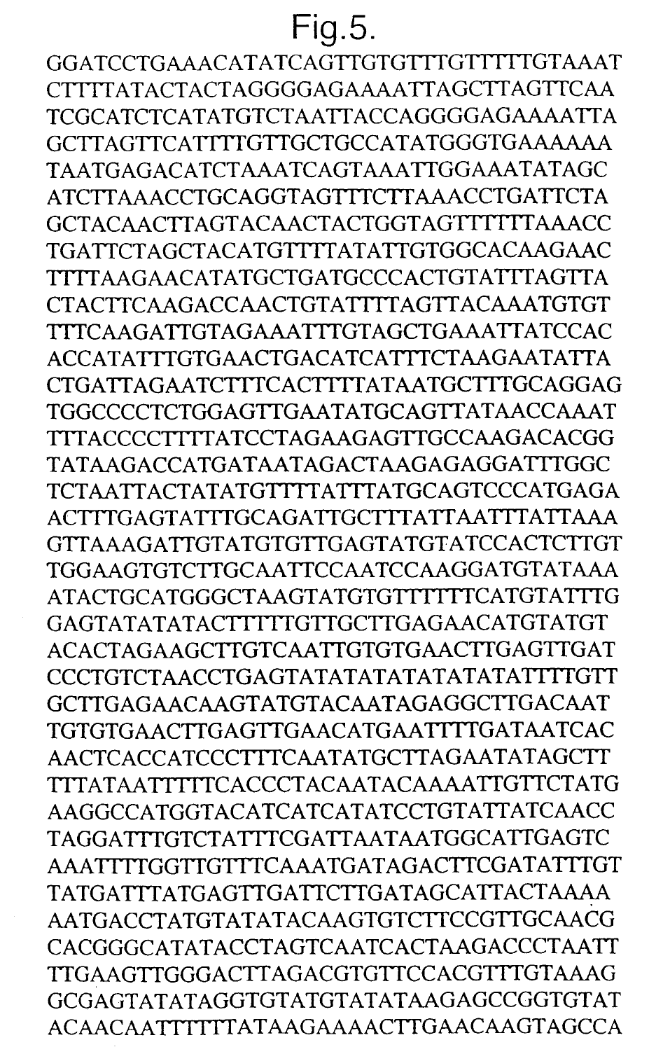

FIG. 4 shows

(A) Physical map of the ZmC5::uidA construct and junction sequence. The A residues by*, [symbol] and ♦ correspond to the ZmC5 transcription start point, the T to A mutation to remove the Hind III site (for ease of cloning) and the 3′ terminus of the ZmC5 promoter region, respectively. The uidA translation start is underlined.

(B) Histochemical analysis of GUS activity in pollen from transgenic ZmC5::uidA tobacco showing segregation of the blue staining.

(C) Promoter activity of ZmC5 in transgenic tobacco tissues from two transgenic lines. GC5-2 (unfilled columns) and GC5-7 (filled columns). Data from each line represents the mean from two independent assays, normalised to a non-transgenic control. Bud stages are as follows: Bud-1 corresponds to buds of 5-8 mm. (microspores at meiosis/tetrad stage), Bud-2: buds of 10-12 mm (uninucleate microspores). Bud-3: 13-15 mm, (microspore mitosis). Bud-4: (17-22 mm) early- to mid-stage binucleate gametophyte, Bud-5: (27-45 mm) mit- to late stage binucleate gametophyte (Tebbutt et al.., supra.).

FIG. 5 shows the DNA sequence encoding the ZmC5 promoter sequence in maize. The underlined A is the putative transcriptional start point and the bold and underlined ATG is the translational start point.

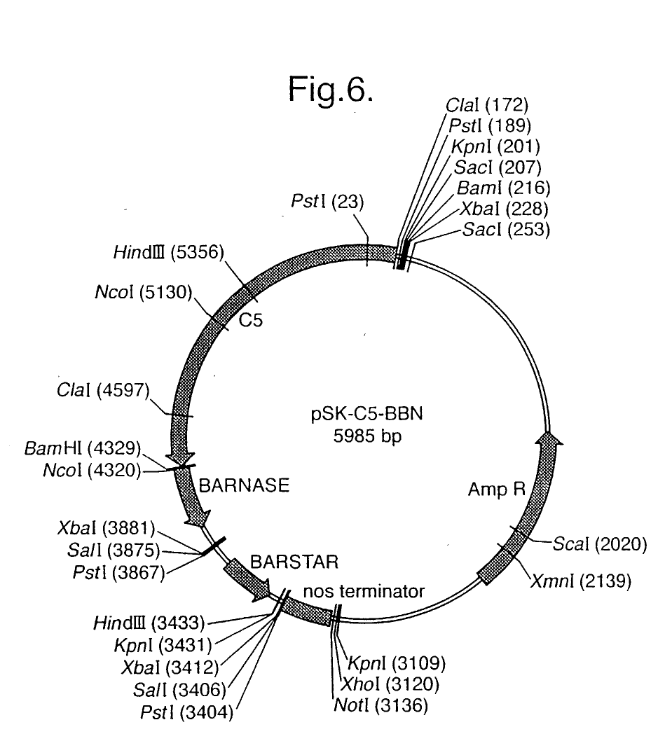

FIG. 6 shows an expression cassette comprising C5-barnase/barstar-nos.

### EXAMPLE 1

### ZmC5 cDNA and Genomic Clones

Maize (inbred line A 188) pollen and germinating pollen (H J Rogers et al.., Plant J4 (1993) 875-882) cDNA libraries were screened using cDNA probes made to pollen and shoot poly(A)⁻ RNA following standard procedures (FM Ausubel et al.., Current protocols in Molecular Biology, Wiley, N.Y. (1990)). All clones, totalling 1,101, that showed hybridisation to the radiolabelled pollen cDNA but not to the radiolabelled shoot DNA were picked. Random colonies were picked and sequenced at the 5′ end and the sequence compared on current databases. One clone which had an insert of 800 bp showed significant sequence identity with known pectin methyl esterases. The pollen cDNA library was re-screened with this cDNA insert and the full length ZmC5 clone (ZmC5c) was identified and sequenced.

A maize (inbred line B73) genomic library (8×10⁶ plaques) was screened using a PCR fragment corresponding to the 5′ 270 bp of cDNA clone ZmC5c labelled by random priming (Ausubel et al.., supra). One positive clone ZmC5 g was plaque purified. Comparison of the sequence of a 2.5 kb SalI fragment of ZmC5 g subcloned into pUC19 with ZmC5c (and deposited as NCIMB 40915) showed that the two sequences overlapped and that they were identical, indicating that the clones represent the same gene. A representation of the overlap between the 2.5 kb fragment of the genomic clone with the cDNA is shown in FIG. 1.

A transcriptional start point was mapped using two oligonucleotide primers complementary to nucleotides 1 to 21 (5′- ACCTAGGAGAGCCTTTGCCAT-3′) and 56 to 82 (5′-AGCGGGTGACGGTGGCGACCACACCGA-3′) of the coding sequence (data not shown). The products unequivocally locate the transciptional start point on the A nucleotide at only 15 bases upstream of the putative ATG (FIG. 1). Other pollen-specific genes have been found to have long 5′-untranslated sequences (S J Tebbutt et al.., Plant Mol. Biol. (1994) 25: 283-299). Thus this region of ZmC5g appears to be unusually short. The calculated free energy for this short 5′ UTL is of 0.9 kJ/mol(M. Zuker, Meths Enzymol. (1989) 180: 262-288) making it unlikely that it is able to form any stable secondary structure.

An open reading frame of 1692 bp was identified and the putative ATG start codon (FIG. 1) fits well with the consensus sequences (H A Lutcke et al.., EMBO J 6: (1987) 43-48). A putative TATAA motif (C P Joshi, Nucl. Acids Res. 15:6648-6653 (1987)) starts at −32, however no recognisable CAAAT motif is found in the 60 bp upstream from the transciptional start (FIG. 1) as has been found in many other plant genes. ZmC5c lacks a clearly recognisable AATAAA polyadenylation site signal. This is not unusual: 30% of plant genes lack a recognisable AATAAA motif (B D Mogen et al.., Plant Cell 2 (1990) 1261-1272). In ZmC5c, a stretch of five A residues 16 bp upstream from the poly A addition site may be acting as a polyadenylation signal, although other pollen specific transcripts have been found to have longer than average distances between the AATAAA motif and the poly(A)⁺ addition site (Tebbutt et al.., supra.)

### EXAMPLE 2

### Deduced Amino-Acid Sequence of ZmCS

The predicted amino acid sequence of ZmC5 (563 amino acids) was compared to the EMBL and GenBank databases, and revealed a high degree of homology to both plant (between 30.9% and 41.4%) and microbial PMEs (between 18.6% and 20.8%). An alignment of amino acid sequences showed conservation across both plant and microbial sequences restricted primarily to the C-terminal end of the protein which includes four regions likely to be the catalytic domains or active sites of the enzyme (D. Albani et al.., Plant Mol Biol (1991) 16:501-513),O Marcovic et al.., Protein Sci 1 1288-1292 (1992)). In vitro mutagenesis of the A niger PME (B Duwe et al., Biotechnol. Letts 18: 621-626 (1996)) indicated that a histidine residue, which is conserved in ZmC5. within the region I may be located at the active site of the enzyme, and in A. niger is required for enzyme activity. However this histidine is replaced by other amino acid residues in several PMEs of both plant and fungal origin, suggesting that is it not essential in all PMEs. In a comparison of the plant PMEs, ZmC5 shows a closer relationship to the P inflata ‘late’ pollen expressed PPF gene (J H Mu et al.., Plant Mol Biol 25: 539-544 (1994)), than to B. napus ‘early’ pollen expressed Bp19 gene (D. Albani et al.. supra.)

### EXAMPLE 3

### Estimation of ZmC5 Gene Number

A maize (inbred line A188) genomic Southern blot containing 15 μg of DNA digested with either BamHI, EcoRI, or Hind III was probed with radiolabelled full-length ZmC5 cDNA insert. Two strong hybridising bands in each lane of the blot in FIG. 2 suggests the presence of at least two similar genes in the maize genome. Several further bands with show a much weaker signal suggests that this gene family may also comprise several less related members.

### EXAMPLE 4

### Spatial and Temporal Expression of ZmC5

A northern blot containing 10 μg total RNA from eight maize tissues was probed with the cDNA ZmC5c to determine the expression programme of the gene. A transcript of approximately 2.0 kb was detected only in pollen and germinating pollen (FIG. 3(A)), indicating that, within the limits of detection of this technique, expression of this gene appears to be restricted to these two tissues. No signal was detectable in leaf, root, shoot, cob, endosperm or embryo. The expression programme during spikelet development was also determined. FIG. 3(B) shows a Northern blot containing total RNA from 0.25, 0.5 and 1.0 cm spikelets, mature pollen and germinating pollen. The ethidium bromide stained gels demonstrate the equal loadings of RNA in the lanes of each gel.

Spikelets were staged by staining the anthers with acto-carmine, and the anthers were found to contain cells at the following stages: pre-meitotic sporpogenous cells (0.25 cm), mid-prophase I (0.5 cm), maturing pollen grains (1.0 cm). Some overlap between consecutive stages is however inevitable due to the variation in the developmental stage between the two florets within the same spikelet. This Northern analysis shows that ZmC5 expression is restricted to mature dehisced pollen and germinating pollen with no detectable expression in any other maize tissues including spikelets containing cells in earlier stages of microsporogenesis.

### EXAMPLE 5

### Expression of ZmC5 Promoter/GUS Constructs in Transgenic Plants

Transciptional fusions were made between the 5′ region of ZmC5g and the reporter gene β-glucuronidase (FIG. 4A), and used to transform tobacco by Agrobacterium transformation. The construct was made as follows:—the two Sph I sites, one within the 2.5 kb SalI fragment which contains 2 kb of 5′ sequence relative to the ATG on one within the polylinker, were used to remove the 3′ end from position −61 to +403 (FIG. 1). This was directly replaced by and Sph I digested PCR fragment that included the region −61 to +1, additional restriction sites positioned at the 3′ end (Bam HI, Hind III, Sal I) and a mutation to remove the Hind III site positioned at the transcriptional start (FIGS. 1 and 4A). The original 5′ Sal I site and the introduced 3′ sal I site were then used to excise the ZmC5 promoter region which was cloned into the Sma I site of pGUS. The ZmC5 promoter-UID-A transcriptional fusion was then transferred to pBin 19 vector (FIG. 4A) and used for Agrobacterium-mediated leaf-disc transformation of Nicotiana tabacum var Samsun. Transformants were selected on kanamycin and primary transgenic plants were regenerated, two of which, positive for expression of the transgene, were taken to the T2 generation.

Pollen grains from dehisced anthers of the transgenic plants were harvested and stained for GUS activity as described by J. A. Jefferson (Plant Mol Biol Rep (1987) 5: 387-405). Two plants were positive showing approximately 50% blue staining pollen (FIG. 4B). No blue colouration was detected in non-transgenic controls. To investigate the number of integration sites, plants from two transgenic lines were selfed, and progeny were scored for resistance to kanamycin. Of the progeny assessed from transgenic plant GC5-2, 303 were kanamycin resistant and 8 kanamycin sensitive giving a mean ratio of 38:1 indicative of at least two integration sites The progeny of transgenic plant GC5-7 gave a mean ratio of kanamycin resistant to kanamycin sensitive of 3.8:1 indicative of a single integration site (expected ratio for one integration site is 3:1, for two, 15:1 and for three 63:1).

Extracts were made from a range of tissues including five stages of developing anthers, and analysed fluorimetrically fir GUS expression (Jefferson. supra.) FIG. 4(C) shows GUS activities from two transgenic plants. Only very low levels of expression are detectable in tissues other than developing and mature dehisced anthers. In tobacco the stage of bud development can be correlated with bud length (Tebutt et al.., supra.) but this is dependent on the growth conditions. Thus Bud-1 corresponds to microspores at mitosis/tetrad stage, Bud-2, uninucleate microspores, Bud-3 microspore mitosis, Bud-4, early to mid-stage binucleate gametophyte, Bud-5 mid- to late-stage binucleate gamteophyte. Microspore stages as assessed by DAPI staining (data not shown) indicate that the timing of expression of the ZmC5 promoter in tobacco agrees well with its expression in maize based on the Northern data (FIG. 3; FIG. 4C). Thus both in it native environment in maize and in transgenic tobacco, the ZmC5 promoter function late in pollen development and is virtually inactive before microspore mitosis. Some variation in expression can be noted between the two transgenic lines with the GC5-2 showing higher levels of expression in most tissues tested. Variation in expression levels are commonly found in transgenic populations and have been ascribed to the site of insertion of the transgene 9SLA Hobbs et al.., Plant Mol Biol 21: 17-26 (1993)).

### EXAMPLE 6

### Expression of GUS in Pollen Driven by AlcA Inducible Promoter

A plant transformation vector comprising the AlcA promoter driving expression of GUS and a 35S CaMV promoter driving the expression of AlcR has been introduced into tobacco and tomato plants. GUS expression may be studied in all tissues before and after induction with ethanol as a root drench.

GUS staining of tomato anthers and pollen shows clear expression of GUS after induction. The same result is expected from pollen from other species.

### EXAMPLE 7

### Preparation of C5-Barnase Cassette—A Dominant Gametophytic Male_Sterility Cassette.

The unique SalI site of pBluescipt SK+ (Stratagene) was replaced with a NotI recognition site by insertion of the an oligonucleotide linker MKLINK4 (5′-TCGATTCGGCGGCCGCCGAA-3′) into the digested SalI site. A 0.9 kb, BamHI-HindIII fragment carrying the coding region of barnase followed by a bacterial-promoter-driven barstar coding region, was inserted into the corresponding fragment of the modified pBluescript. The nos terminator on a HindIII-NotI fragment was inserted into the corresponding fragment of the resulting vector. An unwanted BamHI site was then removed using Stratagene's QuickChange system, following the manufacturer's instructions and using oligonucleotides DAM-3A (5′-GGTCGACTCTAGAGGAACCCCGGGTACCAAGC-3′) and DAM-3S (5′- GCTTGGTACCCGGGGTTCCTCTAGAGTCGACC-3′). The resulting plasmid (named pSK-BBN) was digested to completion with BamHI, dephosphorylated with shrimp alkaline phophatase (37° C., 1 hour). A 1.9 kb BamHI fragment of the C5 5′ flanking region was ligated into this, followed by digestion with BamHI and PstI to check for presence and orientation of the insert, respectively. The resulting plasmid was named pSKC5-BBN (FIG. 6). The entire cassette is then removed as an EcoRI-NotI fragment to a binary plant transformation vector pVB6. The construct is then introduced into Agrobacterium Tumefaciens by the freeze-thaw method. Standard techniques are used to introduce the DNA into tobacco.

### EXAMPLE 8

### Analysis of Sterile Transgenic Plants

Primary transformants are selected by growth on kanamycin in tissue culture and this confirmed by PCR analysis. The plants are grown to maturity in the glass house. Pollen is collected from anthers after dehiscence and a vital stain is used to establish whether the pollen is fertile or sterile. 50% of the pollen is expected to be sterile. Backcrossing these plants with wild type plants (after anther removal) or allowing self pollination to occur results in progeny in which 50% of pollen is sterile.

Other modifications to the present invention will be apparent to those skilled in the art without departing from the scope of the invention.

1. A recombinant nucleic acid sequence which comprises a promoter sequence of the ZmC5 gene in maize, or a variant or fragment thereof, which acts as a promoter in pollen.

2. A recombinant nucleic acid sequence according to claim 1 which comprises approximately 2 kb upstream of the transcriptional start site of the ZmC5 gene of maize as shown in FIG. 1.

3. A recombinant nucleic acid sequence according to claim 1 or claim 2 which comprises a promoter sequence comprising at least part of the DNA sequence as shown in FIG. 5 or at least part of a sequence which encodes a promoter which has substantially similar activity to that shown in FIG. 5, or a variant or fragment thereof.

4. An expression cassette comprising a nucleic acid sequence according to any one of claims 1 to 3 wherein the cassette is arranged to control a gene which it is desired to be expressed in pollen and which encodes a product capable of having an impact on pollen production, insecticidal toxins, or enhances or modifies the nutritional value of pollen.

5. An expression cassette according to claim 4 wherein said gene comprises a gene which is deleterious to pollen development.

6. An expression cassette according to claim 5 wherein said gene comprises a gene encoding either barnase, adenine nucleotide translocator, mutant tubulins, T-urf or trehalose phosphate phosphatase (TPP) or a ribozyme.

7. An expression cassette according to claim 4 wherein the said gene comprises a selectable marker gene.

8. An expression cassette according to claim 7 wherein the selectable marker gene comprises and antibiotic resistance gene.

9. An expression system comprising an expression cassette according to any one of claims 4 to 8.

10. An expression system according to claim 9 which comprises a gene which is deleterious to pollen, and which further comprises an expression cassette comprising a second nucleic acid sequence which encodes a peptide or protein able to overcome the effect of said deleterious gene, operatively interconnected with a chemical inducible promoter.

11. An expression system according to claim 10 wherein the said nucleic acid sequence comprises a restorer gene.

12. An expression system according to claim 11 wherein the said restorer gene is barstar or TPS.

13. An expression system according to claim 10 wherein said nucleic acid sequence encodes a construct which is sense or antisense to said deleterious gene so as to suppress expression thereof.

14. An expression system according to claim 10 wherein said second nucleic acid sequence encodes a repressor protein to lac, tet, 434, lac-His, which interacts with an operator sequence which is operably linked to the nucleic acid sequence of any one of claims 1 to 3, or an Amplicon™, so as to prevent expression of the first gene which it is desired to be expressed in pollen.

15. An expression system according to any one of claims 10 to 14 wherein the inducible promoter is the AlcA/R, GST or Ecdysone inducible promoter.

16. An expression system according to any one of claims 9 to 15 which further comprises a selection marker.

17. An expression system according to any one of claims 9 to 16 wherein the gene which it is desired to be expressed in pollen is linked to a herbicide resistance gene.

18. An expression system comprising
(a) a first promoter sequence which is expressed specifically in pollen;
(b) a first gene which when expressed, disrupts pollen biogenesis, under the control of said pollen specific promoter;
(c) a second promoter sequence responsive to the presence or absence of an exogenous chemical inducer; and
(d) a second gene encoding an element which can inhibit either expression of said first gene, or can inhibit the said first protein, operably linked to and under the control of said second promoter sequence.

19. A method of producing a plant which method comprises transforming a plant cell with an expression system according to any one of claims 9 to 18.

20. A plant cell which comprises an expression system according to any one of claims 9 to 18.

21. A plant which comprises cells according to claim 20.

22. A method of inducing male sterility in plants which method comprises transforming a plant with an expression system according to any one of claims 10 to 18.

23. A method of controlling the fertility of a plant which comprises transforming said plant with an expression system according to claim 11 or claim 18, and when fertility is required to be restored, activating the inducible promoter.

24. A method according to claim 23 wherein the inducible promoter is activated by application of a chemical to the plant.

25. A replicable viral RNA vector (Amplicon™) which comprises a recombinant nucleic acid according to any one of claims 1 to 3.

26. A method of transforming pollen which comprises transforming pollen cells with an expression system according to claim 9 or claim 18.

27. A recombinant nucleic acid, an expression cassette, an expression system or a method substantially as hereinbefore described with reference to any one of the accompanying Figures.


SEQUENCE LISTING (9 sequences)

SEQ ID NO: 1 - DNA, 90 bp, Artificial Sequence
  ZmC5 cDNA
gcatgcatccacgtccgtacgcagccatttcgtctataaatttgcttcccatccgattcaactacaagcttgcgggcaaaaatggcaaag

SEQ ID NO: 2 - DNA, 55 bp, Artificial Sequence
  ZmC5::uidA construct and junction sequence
actacaaggtaccgggcacccatccaagcttgtcgaggtaggtcagtcccttatg

SEQ ID NO: 3 - DNA, 1890 bp, Artificial Sequence
  ZmC5 DNA promoter sequence in maize
ggatcctgaaacatatcagttgtgtttgtttttgtaaatcttttatactactaggggagaaaattagcttagttcaatcgcatctcatatgtctaattaccaggggagaaaattagcttagttcattttgttgctgccatatgggtgaaaaaataatgagacatctaaatcagtaaattggaaatatagcatcttaaacctgcaggtagtttcttaaacctgattctagctacaacttagtacaactactggtagttttttaaacctgattctagctacatgttttatattgtggcacaagaacttttaagaacatatgctgatgcccactgtatttagttactacttcaagaccaactgtattttagttacaaatgtgttttcaagattgtagaaatttgtagctgaaattatccacaccatatttgtgaactgacatcatttctaagaatattactgattagaatctttcacttttataatgctttgcaggagtggcccctctggagttgaatatgcagttataaccaaattttaccccttttatcctagaagagttgccaagacacggtataagaccatgataatagactaagagaggatttggctctaattactatatgttttatttatgcagtcccatgagaactttgagtatttgcagattgctttattaatttattaaagttaaagattgtatgtgttgagtatgtatccactcttgttggaagtgtcttgcaattccaatccaaggatgtataaaatactgcatgggctaagtatgtgttttttcatgtatttggagtatatatactttttgttgcttgagaacatgtatgtacactagaagcttgtcaattgtgtgaacttgagttgatccctgtctaacctgagtatatatatatatatattttgttgcttgagaacaagtatgtacaatagaggcttgacaattgtgtgaacttgagttgaacatgaattttgataatcacaactcaccatccctttcaatatgcttagaatatagctttttataatttttcaccctacaatacaaaattgttctatgaaggccatggtacatcatcatatcctgtattatcaacctaggatttgtctatttcgattaataatggcattgagtcaaattttggttgtttcaaatgatagacttcgatatttgttatgatttatgagttgattcttgatagcattactaaaaaatgacctatgtatatacaagtgtcttccgttgcaacgcacgggcatatacctagtcaatcactaagaccctaattttgaagttgggacttagacgtgttccacgtttgtaaaggcgagtatataggtgtatgtatataagagccggtgtatacaacaattttttataagaaaacttgaacaagtagccaggtgttgaaatcttcatatatgtgccgacgccattcaacatcatatttggcttctggcgaggatcgtagtatcaagcaacataaaagcaatgacaaacagcgaagcacaaagatctcccaggctcgtcataaactaatcacaatgttgtttgtcctccacaattagcacaacccattttagaaaaagatgccacgatcgatcgagacgttggccagctatcaaacagataagaactacccaaatatttcctaaatccagaacggaagacccattgactaggtccttacctctcaaatagacagactattcttctccacatcaaaatatagggactcccgatgcaacaaacacgggccaccacacaacaatggtgaaatgaccatgcatgcatccacgtccgtacgcagccatttcgtctataaatttgcttcccatccgattcaactacaagcttgcgggcaaaaatggcaaaggctc

SEQ ID NO: 4 - DNA, 21 bp, Artificial Sequence
  oligonucleotide primer
acctaggagagcctttgccat

SEQ ID NO: 5 - DNA, 27 bp, Artificial Sequence
  Oligonucleotide primer
agcgggtgacggtggcgaccacaccga

SEQ ID NO: 6 - protein, 6 aa, Artificial Sequence
  Oligonucleotide linker
MKLINK

SEQ ID NO: 7 - DNA, 20 bp, Artificial Sequence
  Oligonucleotide linker
tcgattcggcggccgccgaa

SEQ ID NO: 8 - DNA, 32 bp, Artificial Sequence
  Oligonucleotide DAM-3A
ggtcgactctagaggaaccccgggtaccaagc

SEQ ID NO: 9 - DNA, 32 bp, Artificial Sequence
  Oligonucleotide DAM-3S
gcttggtacccggggttcctctagagtcgacc